(19) United States
(12) Patent Application Publication
(10) Pub. No.: US 2026/0076620 A1
(43) Pub. Date: Mar. 19, 2026

(54) SYSTEMS AND METHODS UTILIZING PHYSIOLOGICAL SIGNALS TO ALTER BEHAVIOR

(71) Applicant: Arizona Board of Regents on Behalf of the University of Arizona, Tucson, AZ (US)

(72) Inventors: Kathryn E. R. Kennedy, Tucson, AZ (US); Michael Grandner, Tucson, AZ (US); Fabian Fernandez, Tucson, AZ (US); Ann Skulas-Ray, Tucson, AZ (US)

(21) Appl. No.: 19/330,086

(22) Filed: Sep. 16, 2025

Related U.S. Application Data
(60) Provisional application No. 63/695,135, filed on Sep. 16, 2024.

(51) Int. Cl. A61B 5/00; A61B 5/0531; A61B 5/16; G06F 3/01

(52) U.S. Cl. CPC A61B 5/486; A61B 5/0531; A61B 5/165; A61B 5/4836; A61B 5/7275; G06F 3/015

(57) ABSTRACT

Systems for altering a subject's behavior through a user interface are described herein. Altering a subject's behavior change is done through detecting a signal from a subject indicating that a subject is about to engage in a behavior and initiating a real-time intervention through a user interface before the subject engages in the behavior.

### CROSS-REFERENCE TO RELATED APPLICATIONS

This application claims the benefit of and priority to U.S. Application No. 63/695,135, filed Sep. 16, 2024, which is hereby incorporated herein by reference in its entirety.

### FIELD OF THE INVENTION

The disclosed invention is generally in the field of behavior altering, specifically in the area of systems and methods for altering behavior such as resource consumption (e.g., food and drinks) and/or physical activity (e.g., walking, running, sleeping, weightlifting, etc.).

### BACKGROUND OF THE INVENTION

People may desire to change their behavior for improved health, happiness, well-being, performance, or other goals. Often, a person may have a goal that involves many aspects of that person's life and are difficult to track or to change. For example, a person may desire to improve sleep quality, exercise more, adhere to a prescribed medication, improve diet, decrease cumulative screen time on various devices, spend more time with family or friends, spend more time doing homework, be more focused while working, have longer attention spans, or achieve other behavior outcomes. However, in today's complex technological environment with multiple distractions and opportunities for circumventing behavior restrictions make it a significant challenge to change behavior to achieve a desired goal. Accordingly, there remains an unmet need to develop improved systems to assist individuals alter a behavior associated with a desired goal.

Therefore, it is an object of the invention to provide systems and/or methods to assist individuals alter behavior with real-time intervention.

### SUMMARY OF THE INVENTION

Described herein are systems having at least one sensor and at least one processing unit for altering a behavior that a subject (e.g., an individual) desires to be altered. The system alters behavior by processing one or more signals collected by at least one sensor and providing real-time intervention, before the subject engages in the behavior.

Also described are methods of using the disclosed systems for altering a behavior that a subject desires to be altered. An external stimulus, internal stimulus, or a combination thereof, prompts a subject and subconsciously evokes a behavior. This subconscious trigger sets in motion a cascade of biological mechanisms in the subject, which results in one or more signals that are detected and/or recorded by at least one sensor. The one or more signals are then processed by one or more processing units operably linked to the sensor. The ultimate endpoint is the provision of a real-time intervention to the subject through a user interface or another individual before the subject engages in the behavior that is to be altered.

The behavior altered by the system can be internet usage, screen time, video game usage, social media usage, exercise pattern, diet behavior (e.g., eating and/or drinking behavior), sleep pattern, a medication adherence, or any other behavior. In a preferred form, the behavior altered by the system is diet/eating.

The sensors for collecting the signals can be electrodermal sensors, accelerometers, gyroscopes, magnetometers, image sensors, cameras, optical sensors, proximity sensors, pressure sensors, odor sensors, gas sensors, Global Positioning Systems (GPS) circuit, microphones, thermometers, ambient light sensors, UV sensors, electrodes for electromyographic (“EMG”) potential detection, bio-impedance sensors, spectrometers, glucose sensors, touchscreen or capacitive sensors. In some preferred forms, the sensor includes an electrodermal sensor. In some forms, the electrodermal sensor is in the form of a wrist-wearable device (e.g., a wrist-wearable bracelet) or a finger-wearable device (e.g., a finger-wearable ring). Preferably, the sensor is in the form of a wearable finger-worn ring.

The one or more signals collected by the at least one sensor can be physiological signals. The physiological signals can be galvanic skin response signals, motion data, temperature, heart rate, pulse, blood or body chemistry, and audio or video recording. Preferably, the physiological signal is a galvanic skin response signal and/or heart rate.

Processing units for processing the signals can be, for example, a computing processor. In some forms, the computer processor can be a microprocessor, a single-core processor or a multiple-core processor, a single-core processor configured with virtual processing technologies, logical processors to simultaneously execute and control multiple processes. In some forms, the processing units may implement technologies such as virtual-machine technologies to execute, control, run, manipulate, and/or store multiple processes, applications, and/or programs. In some forms, the processing unit may include a multiple-core processor configured to provide parallel processing functionalities for execution of multiple processes simultaneously. In some forms, the processing unit can be configured, for example, to execute one or more instructions stored in memory to perform operations. In some forms, the processing unit is in the form of a laptop, computer, server, tablet, or a smartphone.

A real-time intervention provided by the processing unit can be an alert, message, notification, or a combination thereof, making the subject aware that the subject is about to engage in a specific behavior; a directive that the subject engages in another specific behavior; a reminder of goals previously set; or a combination thereof. In some forms, the real-time intervention is psychological intervention. In some forms, the psychological intervention is a mindfulness-based intervention.

Aspects consistent with the present disclosure may include real-time intervention to alter a behavior that a subject desires to be altered. A subject (e.g., an individual) can be a member of a group (e.g., a family, a team, employees, co-workers, members of an organization, members of a community, a group of friends, a class, etc.). The real-time intervention can be managed by the individual himself or herself. The real-time intervention can be managed by an administrator (e.g., a group leader, a coach, an employer, a parent, a caretaker, a friend, a support person, etc.).

Aspects consistent with the present disclosure can include real-time intervention to alter a behavior that a group, as a whole, desires to be altered. For example, a real-time intervention can relate to a group measurement goal in regard to a limit on the total number of times two or more members of a group (e.g., family members) engage in a particular behavior without setting a measurement goal for individual members doing that activity. As another example, an intervention may relate to a group measurement goal of a percentage of compliance with individual measurement goals.

The system also includes a user interface operably linked to one or more processing units in the system. In some forms, the real-time intervention is provided via the user interface. In some forms, the user interface is a graphical user interface (GUI), menu-driven interface, command line interface, conversational user interface, touch user interface, voice user interface, form-based user interface, natural language user interface, and a mobile user interface. In some preferred forms, the user interface is a GUI. In some forms, the GUI is in the form of a laptop, computer, tablet, or a smartphone.

### BRIEF DESCRIPTION OF THE DRAWINGS

FIG. 1 is a diagram showing an exemplary system for guiding the change of eating behavior with galvanic skin response data collection and mindfulness-based intervention.

FIG. 2A-2C are exemplary graphs showing the galvanic skin response in a single participant over the course of approximately 48 h. The food symbols indicate a major eating episode occurred, to demonstrate proximity to a galvanic skin response peak.

### DETAILED DESCRIPTION OF THE INVENTION

### I. Definitions

The term “behavioral intervention,” or simply, an “intervention” as used herein includes a variety of interventions that are designed specifically to increase health, physical, psychological, and/or emotional well-being of a subject and provide mechanisms to assist the subject alter a behavior the subject desires to be altered. In accordance with the present disclosure, an “intervention” may simply be an activity, based on prior evidence-based research, showing that when a subject engages with the activity (as intended), the subject benefits in terms of his or her health, physical, psychological, and/or emotional well-being. In accordance with the present disclosure, a system provides a user with an intervention. Generally, this terminology is intended to mean that the system provides and/or loads an intervention, and commences and/or engages the subject in a set of activities. An intervention includes a set of activities or verbal instructions or tasks to be carried out or otherwise performed either by the subject or between the subject and a coach (or a virtual coach) or individuals in a group. An intervention also generally has a purpose of activating certain mental or physical mechanisms within the user's mind and/or body, by bringing out certain emotional reactions from the user. As such, an intervention generally comes with an intended implementation, that is, a specific method or approach intended by a creator of such intervention for the set of activities to be carried out in order to most efficiently achieve the underlying purpose behind the intervention. The intended implementation may come in the forms of criteria, conditions, requirements, or factors that are each designed to be met by the user by performing a specific act or speaking a specific word. Accordingly, the most effective and efficacious way to advance an intervention is for the user to stay faithful to the intended implementation through the course of the intervention.

The term “Operably linked”, refers to the connection of at least two components in a CIS via technology including, but not limited to, ethernet, Bluetooth, near field communication, WiFi, integrated circuits, or a combination thereof.

The term “real-time” refers to a system in which a signal is transmitted to a user of the system, within 1, 2, 3, 4, 5, 10, 15, 20, or no more than 30 minutes after the system receives and/or detects an input signal. The transmitted signal can be a processed signal derived from processing the input signal.

### II. Systems

Described herein are systems having at least one sensor and at least one processing unit for altering a subject's behavior. Preferably, one or more interventions to guide a subject to alter the behavior desired to be altered are provided through a user interface. Altering behavior is generally done through detecting a signal from a subject indicative of the subject being about to engage in a behavior, and initiating a real-time intervention through a user interface before the subject engages in the behavior.

In some forms, the systems described herein can effectively alter dieting behavior (e.g., eating and/or drinking behavior) using at least one electrodermal sensor to collect galvanic skin response signals and at least one processing unit to process the galvanic skin response signals and a provide real-time intervention through a graphical user interface before the subject eats; drinks; reaches for food; or reaches for a drink.

### A. Systems for Guiding Behavioral Change

Described is a system for guiding behavioral change in a subject, through detecting a subject's desire with sensor data and initiating an intervention (e.g., mindfulness-based intervention) before the subject's desire directs the subject to engage in a certain behavior. Generally, the system for guiding behavioral change includes at least one sensor, at least one computer, and at least one user interface operably linked to each other. The components of the system can be arranged in any suitable order. Additional details about the system's components are provided below.

### 1. Sensor

The system includes at least one sensor, optionally more than one sensor, operably linked with at least one of the other components of the system to collect real-time sensor data from a subject. A sensor is a device that detects and/or responds to some type of input (e.g., changes) from an environment. A sensor converts one or more inputs such as heat, light, motion, pressure, motion, proximity, sound, electrical fields, magnetic fields, or a substance's concentration, into a signal. Preferably, this signal can be measured, interpreted, and/or acted upon. The sensor can be in any suitable form that allows for it to be used as a wearable device on a subject. For example, the sensor is in the form of a wrist-wearable device (e.g., a wrist-wearable bracelet) or a finger-wearable device (e.g., a finger-wearable ring). Preferably, the sensor is in the form of a wearable finger-worn ring.

A variety of sensors can be used. In some forms, sensors include, but are not limited to, electrodermal sensors, accelerometers, gyroscopes, magnetometers, image sensors, cameras, optical sensors, proximity sensors, pressure sensors, odor sensors, gas sensors, Global Positioning Systems (GPS) circuit, microphones, thermometers, ambient light sensors, UV sensors, electrodes for electromyographic (“EMG”) potential detection, bio-impedance sensors, spectrometers, glucose sensors, touchscreen or capacitive sensors.

In some forms, the sensor can be an electrodermal sensor containing at least two electrodes, each of which is configured to be in contact with a part of the subject's skin (e.g., epidermal region, dermal region, and/or subcutaneous region) and an electronic circuit containing a converter (e.g., an analog-to-digital) and a processor that is configured to execute computer-executable instructions in order to bias said electrodes, to measure the current flowing between pairs of electrodes. In some forms, the real-time sensor data collected are a galvanic skin response.

In some forms, the real-time sensor data includes physiological signals. In some forms, the physiological signals include motion data, temperature, heart rate, pulse, blood or body chemistry, audio or video recording and/or other real-time sensor data depending on the sensor type.

The sensor inputs and outputs can be communicated to a processor wirelessly or via wires, in analog or digital form, intermediated by gating and/or clocking circuits or directly provided.

### 2. Processing Unit

The system includes at least one processing unit, operably linked with at least one of the other components of the system for processing the real-time sensor data collected by the at least one sensor and outputting a real-time intervention.

The one or more processing units for processing the signals can be, for example, one or more computing processors. In some forms, the computer processor is a microprocessor, a single-core processor or a multiple-core processor, a single-core processor configured with virtual processing technologies, logical processors to simultaneously execute and control multiple processes. In some forms, the one or more processing units implement technologies such as virtual-machine technologies to execute, control, run, manipulate, and/or store multiple processes, applications, and/or programs. In some forms, the one or more processing units include a multiple-core processor configured to provide parallel processing functionalities for execution of multiple processes simultaneously. In some forms, the one or more processing units are configured, for example, to execute one or more instructions stored in memory to perform operations. In some forms, the one or more processing units are in the form of a laptop, computer, server, tablet, or a smartphone.

### A. Processing of Signals

In some forms, the sensor is an electrodermal sensor that supplies sensor data with an electrode pair to sample the signals at a suitable frequency. In some forms, the real-time sensor data is filtered in amplitude and frequency to remove aberrant signals.

The signals supplied by the electrical conductivity sensor are sampled and then processed to provide the real-time feedback/intervention.

### 3. Real-time Intervention

The system includes the provision of at least one real-time intervention that is outputted before the subject engages in a specific behavior. The real-time intervention provided by the processing unit can be an alert, message, notification, or a combination thereof, that the subject is about to engage in a specific behavior; a directive that the subject engages in another specific behavior; and/or a reminder of goals previously set. In some forms, real-time intervention is psychological intervention. In some forms, the psychological intervention is a mindfulness-based intervention.

Aspects consistent with the present disclosure may include real-time intervention to change a behavior of a subject (e.g., an individual). The subject can be a member of a group (e.g., a family, a team, employees, co-workers, members of an organization, members of a community, a group of friends, a class). The real-time intervention can be managed by the individual himself or herself. The real-time intervention can be managed by an administrator (e.g., a group leader, a coach, an employer, a parent, a caretaker, a friend, a support person, etc.).

Aspects consistent with the present disclosure may include real-time intervention to change a behavior of a group as a whole. For example, a real-time intervention may relate to a group measurement goal in regard to a limit on the total number of times two or more of a group (e.g., two or more family members) engage in a particular activity without setting a measurement goal for individual family members doing that activity. As another example, an intervention may relate to a group measurement goal of a percentage of compliance with individual measurement goals.

### 4. User Interface

The system may include a user interface for receiving the real-time intervention operably linked with the other components of the system.

In some forms, the real-time intervention is provided to the subject via the user interface. A user interface can be a communication and/or interaction point between a subject (e.g., a human) and a machine, program, or device. It includes one or more aspects that a user can feel (e.g., via vibrations), see, hear, and/or touch to operate a system and/or receive information back. In some forms, the user interface is a graphical user interface (GUI), menu-driven interface, command line interface, conversational user interface, touch user interface, voice user interface, form-based user interface, natural language user interface, and a mobile user interface.

In some preferred forms, the user interface is a GUI. In some forms, the GUI is in the form of a laptop, computer, tablet, or a smartphone. In some forms, the real-time feedback/intervention is in the form of executable program stored in the user interface that engages with the subject.

### B. Exemplary System for Altering Behavior

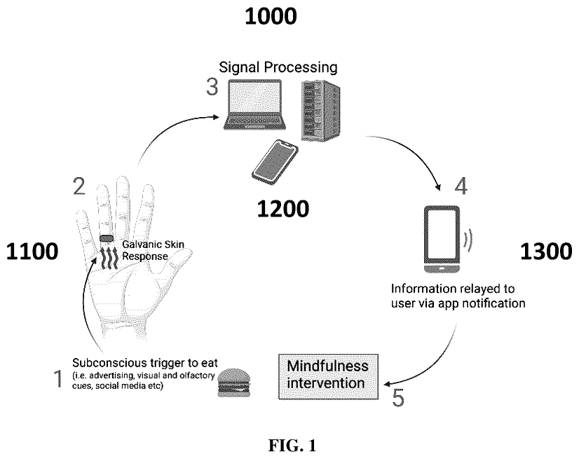

FIG. 1 is a schematic showing an exemplary system for guiding the behavioral change of eating.

As shown in FIG. 1, system 1000 includes a series of components for altering behavior change through real-time intervention, including a galvanic skin response sensor 1100, a processing unit 1200, and a GUI 1300, all of which are operably linked with each other.

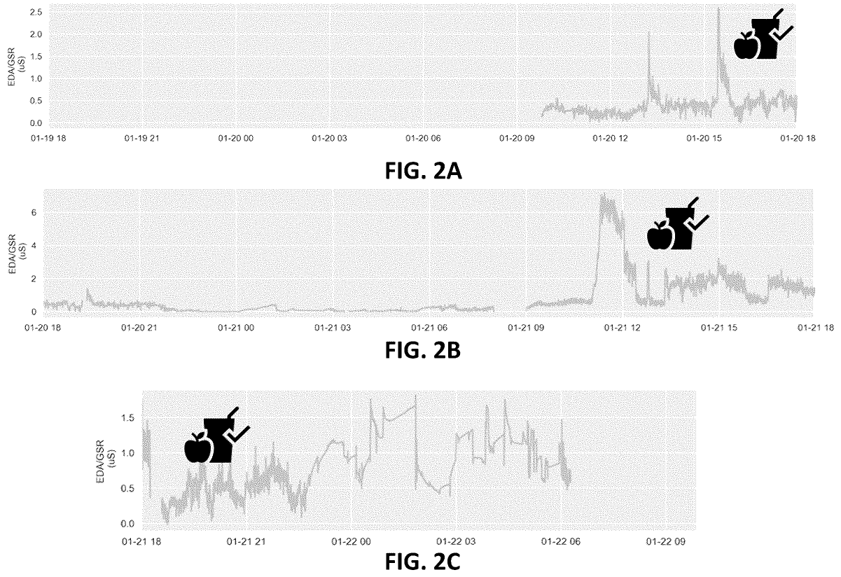

Data showing an increase in a signal (electrodermal activity/galvanic skin response) change before a subject engages in a behavior (eating and/or drinking) are shown in FIGS. 2A-2C. These graphs show the galvanic skin response in a single participant over the course of approximately 48 h. The food symbols indicate a major eating episode occurred, to demonstrate proximity to a galvanic skin response peak.

### II. Method of Using

The systems described herein can be used for altering behavioral change through real-time intervention.

An external or internal stimuli prompts a subject to subconsciously contemplate undergoing a certain behavior. This sets in motion a cascade of signals in the body that eventually results in signals that are recorded by at least one sensor. In some forms, the signals are physiological signals such as elevated skin conductance and/or heart rate. The one or more collected signals are sent to a processing unit where the signals are processed and integrated with each other to output real-time feedback/intervention through a user interface to engage with the subject before the subject undergoes the behavior. In some forms, the user interface is in the form of a GUI.

In some forms, the procedures include a coding function for processing signals (e.g., sensor data). A coding function may include check statements, when statements, while statements, do statements, Boolean logical statements, or the like. Some examples of typical signal processing steps are described in U.S. patent application publication 2023/0371837, the contents of which are hereby incorporated by reference in their entirety.

The disclosed systems and methods can be further understood through the following numbered paragraphs:
1. A system containing a sensor operably linked to one or more processing units configured to process signals from the sensor and provide real-time intervention through a user interface for altering a subject's behavior before the subject engages in the behavior.
2. The system of paragraph 1, wherein the sensor contains electrodermal sensors, accelerometers, gyroscopes, magnetometers, image sensors, cameras, optical sensors, proximity sensors, pressure sensors, odor sensors, gas sensors, Global Positioning Systems (GPS) circuit, microphones, thermometers, ambient light sensors, UV sensors, electrodes for electromyographic (“EMG”) potential detection, bio-impedance sensors, spectrometers, glucose sensors, touchscreen or capacitive sensors.
3. The system of paragraphs 1 or 2, wherein the sensor contains an electrodermal sensor.
4. The system of any one of paragraphs 1 to 3, wherein the real-time intervention contains an alert, message, notification, or combination thereof, that the subject is about to engage in a specific behavior; a directive that the subject engages in another specific behavior; a reminder of goals previously set.
5. The system of any one of paragraphs 1 to 4, wherein the real-time intervention is provided via user interface.
6. The system of any one of paragraphs 1 to 5, wherein the real-time intervention is sent to the subject, another subject, and/or a device.
7. The system of any one of paragraphs 1 to 6, wherein the real-time intervention is an item of digital data representation.
8. The system of any one of paragraphs 1 to 7, wherein the signal contains physiological signals.
9. The system of any one of paragraphs 1 to 8, wherein the signal contains galvanic skin response signals, motion data, temperature, heart rate, pulse, blood or body chemistry, and audio or video recording.
10. The system of any one of paragraphs 1 to 9, wherein the user interface is a graphical user interface (GUI), menu-driven interface, command line interface, conversational user interface, touch user interface, voice user interface, form-based user interface, natural language user interface, and a mobile user interface.
11. The system of any one of paragraphs 1 to 10, wherein the user interface is a graphical user interface (GUI).
12. A method for driving/changing/altering a subject's behavior, the method involving:
(i) obtaining a signal from the subject using a sensor,
(ii) processing the signal using one or more processing units, and
(iii) providing real-time intervention through a user interface to alter the subject's behavior.

We claim:

1. A system comprising a sensor operably linked to one or more processing units configured to process signals from the sensor and provide real-time intervention through a user interface for altering a subject's behavior before the subject engages in the behavior.

2. The system of claim 1, wherein the sensor comprises electrodermal sensors, accelerometers, gyroscopes, magnetometers, image sensors, cameras, optical sensors, proximity sensors, pressure sensors, odor sensors, gas sensors, Global Positioning Systems (GPS) circuit, microphones, thermometers, ambient light sensors, UV sensors, electrodes for electromyographic (“EMG”) potential detection, bio-impedance sensors, spectrometers, glucose sensors, touchscreen or capacitive sensors.

3. The system of claim 1, wherein the sensor comprises an electrodermal sensor.

4. The system of claim 1, wherein the real-time intervention comprises an alert, message, notification, or combination thereof, that the subject is about to engage in a specific behavior; a directive that the subject engages in another specific behavior; a reminder of goals previously set.

5. The system of claim 1, wherein the real-time intervention is provided via user interface.

6. The system of claim 1, wherein the real-time intervention is sent to the subject, another subject, and/or a device.

7. The system of claim, wherein the real-time intervention is an item of digital data representation.

8. The system of claim 1, wherein the signal comprises physiological signals.

9. The system of claim 1, wherein the signal comprises galvanic skin response signals, motion data, temperature, heart rate, pulse, blood or body chemistry, and audio or video recording.

10. The system of claim 1, wherein the user interface is a graphical user interface (GUI), menu-driven interface, command line interface, conversational user interface, touch user interface, voice user interface, form-based user interface, natural language user interface, and a mobile user interface.

11. The system of claim 1, wherein the user interface is a graphical user interface (GUI).

12. A method for driving/changing/altering a subject's behavior, the method comprising:
(i) obtaining a signal from the subject using a sensor,
(ii) processing the signal using one or more processing units, and
(iii) providing real-time intervention through a user interface to alter the subject's behavior.
